(19) United States
(12) Patent Application Publication
(10) Pub. No.: US 2011/0295356 A1
(43) Pub. Date: Dec. 1, 2011

(54) STENT HAVING OPTIMIZED EXPANSION RATIO

(75) Inventor: Chad Abunassar, San Francisco, CA (US)

(73) Assignee: ABBOTT CARDIOVASCULAR SYSTEMS INC., Santa Clara, CA (US)

(21) Appl. No.: 12/789,901

(22) Filed: May 28, 2010

(51) Int. Cl. A61F 2/82

(52) U.S. Cl. 623/1.15

(57) ABSTRACT

An intravascular stent has an elongated tubular member that has a manufactured inner diameter and a manufactured outer diameter. The nominal expansion outer diameter (implanted diameter) is approximately 1.618 times larger than the manufactured outer diameter, which is referred to the Golden Ratio. The Golden Ratio provides for optimal crimping and expansion aesthetics, stent expansion uniformity, and uniform coatability for stent receiving a drug coating.

### BACKGROUND

The invention relates to expandable stents which are adapted to be implanted into a patient's body lumen such as a coronary artery, in order to maintain the patency thereof. Stents are useful in the treatment of atherosclerotic stenosis in the coronary arteries, and other vessels in the body.

Stents are generally tubular-shaped devices which function to hold open a segment of a blood vessel or coronary artery, or other anatomical lumen. They also are useful to support and hold back a dissected arterial lining which can occlude the fluid passageway therethrough. The delivery and deployment of stents in the coronary arteries are well known in the art and various types of catheters are used, along with guide wires, to position and implant a stent in an artery.

Stents typically are formed from thin-walled metal tubing that is laser cut to form a pattern of stent struts in the tubing wall. The stent struts will typically have a generally rectangular cross-section when formed by the laser cutting. One of the difficulties encountered in forming stents having struts with a rectangular cross-section is that the ability to uniformly compress the stent onto the balloon portion of a catheter and expand the stent for implanting into a coronary vessel is not uniform and results in twisting or projecting edges. For example, in U.S. Pat. No. 5,514,154, which is incorporated herein by reference, the struts have an aspect ratio resulting in projecting edges when the stent is expanded and implanted in a coronary artery.

What has been needed and heretofore unavailable is a stent that can be uniformly compressed and expanded without developing out-of-plane twisting of the stent struts, and provide optimal coatability for stent receiving a drug coating.

### SUMMARY OF THE INVENTION

The present invention is directed to a stent formed from an elongated tubular member having struts that form a stent pattern. The struts have a transverse cross-section that is generally rectangular, while the edges may be electro-polished so that the edges are rounded while still maintaining the generally rectangular cross-sectional shape.

In one embodiment, an intravascular stent has an elongated tubular member that has a manufactured inner diameter and a manufactured outer diameter. The manufactured inner diameter and manufactured outer diameter are that of the tubing as received from a supplier and used in a laser cutting process to form struts in the tubing, thereby forming a stent pattern. The elongated tubular member further has a nominal expansion inner diameter and a nominal expansion outer diameter, which approximately represent the diameter of the stent after it has been expanded in a vessel such as a coronary artery. In keeping with the invention, the nominal expansion outer diameter is approximately 1.618 times larger than the manufactured outer diameter. This ratio, sometimes referred to as the Golden Ratio, provides optimal crimping and expansion aesthetics, stent expansion uniformity, and uniform coatability for stents receiving a drug coating. In one embodiment, the elongated tubular member has a radial thickness in the range of 0.0559 mm (0.0022 inch) to 0.1422 mm (0.0056 inch); stent struts having a width in the range of 0.0457 mm (0.0018 inch) to 0.1067 mm (0.0042 inch); a manufactured outer diameter in the range of 1.3208 mm (0.052 inch) to 3.175 mm (0.125 inch); a nominal expansion outer diameter in the range from 2.100 mm (0.0827 inch) to 5.100 mm (0.2008 inch); a nominal expansion inner diameter in the range of 2.010 mm (0.0791 inch) to 5.010 mm (0.1972 inch); and a manufactured inner diameter in the range of 1.2192 mm (0.048 inch) to 3.0734 mm (0.121 inch).

As an example of the application of the Golden Ratio, a tubular member having a manufactured outer diameter of 1.905 mm (0.075 inch) is expanded and implanted in a coronary artery to a nominal expansion inner diameter of 3.0 mm (0.1181 inch). While a 3.0 mm (0.1181 inch) target lesion size is dictated by the patient PCI population (percutaneous coronary intervention), the selection of a 1.905 mm (0.075 inch) initial tubing size lends very closely to a Golden Ratio proportioned nominal deployment size of approximately 2.955 mm (0.1163 inch) inner diameter or 3.045 mm (0.1199 inch) outer diameter. This proportion provides for optimized stent deployment at the majority of PCI lesion sizes.

In one embodiment, the manufactured inner diameter and manufactured outer diameter are adjusted in order to accommodate different lesion diameters. Thus, an elongated tube received from a supplier may have an initial manufactured outer diameter of 1.905 mm (0.05 inch). Prior to using a laser to cut a pattern of struts in the tubing, the diameter of the tubing can be increased by sliding the tubing over a tapered mandrel whereby the taper slightly increases the diameter of the tubing. As an example, the 1.905 mm (0.075 inch) manufactured outer diameter tube can be placed on a tapered mandrel and progressively increase the diameter of the tubing to 2.0828 mm (0.082 inch). When fully expanded in a coronary artery, the nominal expansion outer diameter is approximately 3.243 mm (0.1277 inch). The nominal expansion outer diameter is achieved by multiplying the Golden Ratio of 1.618 times the manufactured outer diameter of 2.0828 mm (0.082 inch) to get 3.243 mm (0.1277 inch). It is noted that if the tube is of substantial length, it may not be possible to increase the tube diameter using the tapered mandrel.

Other features and advantages of the present invention will become more apparent from the following detailed description of the invention when taken in conjunction with the accompanying exemplary drawings.

### BRIEF DESCRIPTION OF THE DRAWINGS

FIG. 1 is an enlarged, partial perspective view of a prior art stent showing a portion of a strut segment projecting radially outwardly after expansion.

FIG. 2 is an elevational view depicting a stent uniformly compressed onto a balloon portion of a catheter and having a crimped diameter.

FIG. 3 is an elevational view depicting an expanded stent on a balloon and having a Golden Ratio expanded and implanted diameter

FIG. 4 is an elevational view of a Golden Ratio expanded stent implanted in a coronary artery.

FIG. 5 is a transverse cross-sectional view depicting an elongated tube having a manufactured inner and outer diameter.

FIG. 6 is a transverse cross-sectional view depicting a stent having a crimped diameter.

FIG. 7 is a transverse cross-sectional view depicting an expanded stent having an expanded inner diameter that is 1.618 times greater than the manufactured inner diameter.

FIG. 8 is a plan view depicting a stent having a manufactured outer diameter.

FIG. 9 is a plan view depicting a stent having a Golden Ratio nominal expanded outer diameter.

FIG. 10 is a plan view, partially in section, depicting a stent having a manufactured diameter sliding onto a tapered mandrel.

FIG. 11 is a plan view, partially in section, depicting a stent having a manufactured diameter sliding onto a stepped diameter mandrel.

### DETAILED DESCRIPTION OF THE PREFERRED EMBODIMENTS

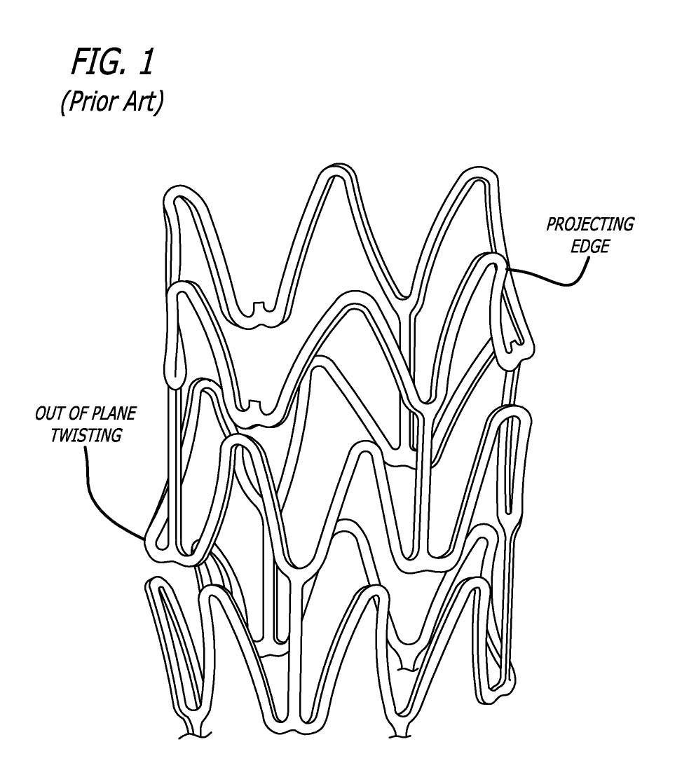

Intravascular stents are generally formed by laser cutting a pattern in a thin walled tube and then etching and/or electropolishing the laser cut stent. This typically produces a stent strut that has a transverse cross-section that is generally square or rectangular with somewhat rounded corners. First generation stent strut cross-sections were generally square in nature, however, clinical trials have shown that thinner struts (less radial thickness) perform better with respect to limiting the formation of restenosis. This conclusion is attributed to the observation that radially thinner stent struts drive a reduction in arterial injury and while also providing reduced disruption of local hemodynamics when compared to radially thicker stent struts. The smaller and thinner stent struts result in less localized strain in the target vessel and therefore produce less injury. Even though thin stent struts provide these clinical benefits, they also must be made stronger and/or stiffer to provided sufficient radial strength and stiffness in order to properly scaffold a target lesion or arterial wall. These thin struts therefore exhibit higher aspect ratios (strut width strut height) greater than one-to-one (square) to provide sufficient bending stiffness to prevent the stent struts from closing due to strut bending loads. In designing a strut with a high aspect ratio, however, the typical strut torsional resistance is relatively low compared to a square (symmetric) cross-section. When torsional resistance is low compared to the bending resistance of a stent strut (which is the case for high aspect ratio struts), the stent strut may twist slightly out of plane when undergoing initial elastic deformation. As shown in FIG. 1, this effect is more severe under large plastic deformation due to compressing or crimping the stent onto the balloon portion of a catheter or during stent expansion into an artery. This localized twisting leads to irregular out-of-plane strut deformations, which are associated with local strut fracture, increased local arterial injury, poor local scaffolding and subsequent plaque prolapse, poor drug delivery (for stents having a drug coating), and an incomplete strut apposition with a potential for associated thrombosis.

In keeping with the present invention, the use of the Golden Ratio in stent design ensures the optimal ratio of nominal stent expansion diameter to laser-cut tubing diameter. More specifically, the present invention design provides uniform stent crimping and expansion which provides the following potential benefits: maximized radial strength and thickness; uniform strut apposition; reduced local vessel injury; improved uniformity of drug delivery; and improved circular expansion. Further, the stent of the present invention is associated with an improved crimp profile, uniformity in crimping, improved stent retention on the balloon portion of a catheter, and more uniform expansion.

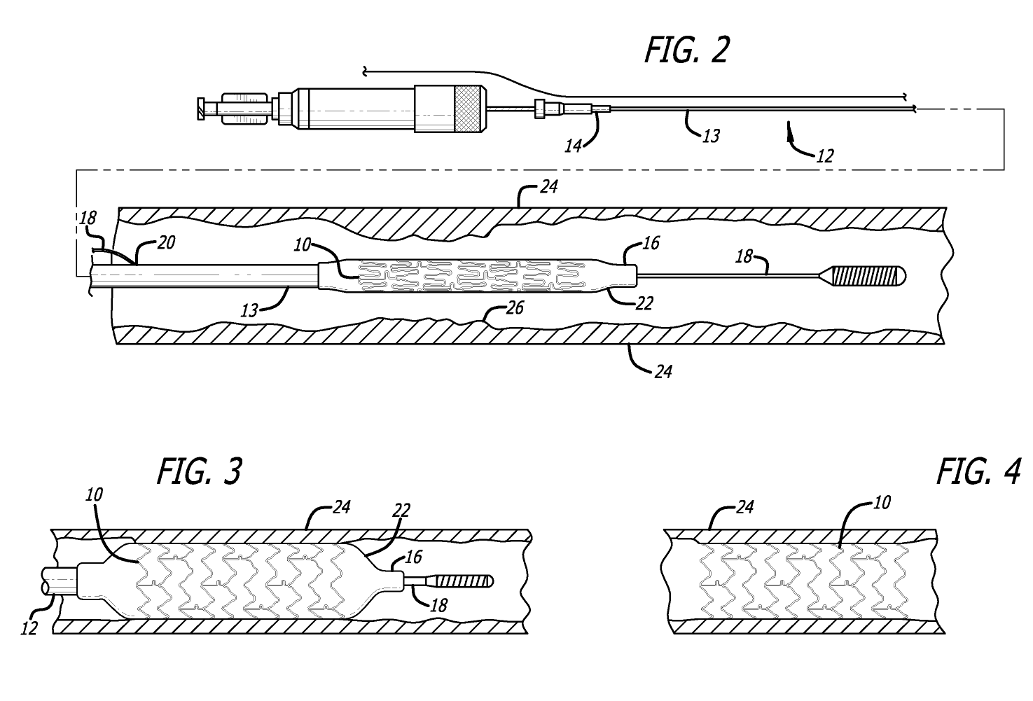

Turning to the drawings, FIG. 2 depicts a Golden Ratio stent 10 mounted on a conventional catheter assembly 12 which is used to deliver the stent and implant it in a body lumen, such as a coronary artery, peripheral artery, or other vessel or lumen within the body. The catheter assembly includes a catheter shaft 13 which has a proximal end 14 and a distal end 16. The catheter assembly is configured to advance through the patient's vascular system by advancing over a guide wire by any of the well known methods of an over-the-wire system (not shown) or a well known rapid exchange catheter system, such as the one shown in FIG. 2.

Catheter assembly 12 as depicted in FIG. 2 is of the well known rapid exchange type which includes an RX port 20 where the guide wire 18 will exit the catheter. The distal end of the guide wire 18 exits the catheter distal end 16 so that the catheter advances along the guide wire on a section of the catheter between the RX port 20 and the catheter distal end 16. As is known in the art, the guide wire lumen which receives the guide wire is sized for receiving various diameter guide wires to suit a particular application. The stent is mounted on the expandable member 22 (balloon) and is crimped tightly thereon so that the stent and expandable member present a low profile diameter for delivery through the arteries.

As shown in FIG. 2, a partial cross-section of an artery 24 is shown with a small amount of plaque that has been previously treated by an angioplasty or other repair procedure. Stent 10 is used to repair a diseased or damaged arterial wall which may include the plaque 26 as shown in FIG. 1, or a dissection, or a flap which are sometimes found in the coronary arteries, peripheral arteries and other vessels.

In a typical procedure to implant stent 10, the guide wire 18 is advanced through the patient's vascular system by well known methods so that the distal end of the guide wire is advanced past the plaque or diseased are 26. Prior to implanting the stent, the cardiologist may wish to perform an angioplasty procedure or other procedure (i.e., atherectomy) in order to open the vessel and remodel the diseased area. Thereafter, the stent delivery catheter assembly 12 is advanced over the guide wire so that the stent is positioned in the target area. The expandable member or balloon 22 is inflated by well known means so that it expands radially outwardly and in turn expands the stent radially outwardly until the stent is apposed to the vessel wall. The expandable member is then deflated and the catheter withdrawn from the patient's vascular system. The guide wire typically is left in the lumen for post-dilatation procedures, if any, and subsequently is withdrawn from the patient's vascular system. As depicted in FIGS. 3 and 4, the balloon is fully inflated with the stent expanded and pressed against the vessel wall, and in FIG. 4, the implanted stent remains in the vessel after the balloon has been deflated and the catheter assembly and guide wire have been withdrawn from the patient.

The stent 10 serves to hold open the artery after the catheter is withdrawn, as illustrated by FIG. 4. Due to the formation of the stent from an elongated tubular member, the undulating components of the stent are relatively flat in transverse cross-section, so that when the stent is expanded, it is pressed into the wall of the artery and as a result does not interfere with the blood flow through the artery. The stent is pressed into the wall of the artery and will eventually be covered with endothelial cell growth which further minimizes blood flow interference. The undulating portion of the stent provides good tacking characteristics to prevent stent movement within the artery. Furthermore, the closely spaced cylindrical elements at regular intervals provide uniform support for the wall of the artery, and consequently are well adapted to tack up and hold in place small flaps or dissections in the wall of the artery.

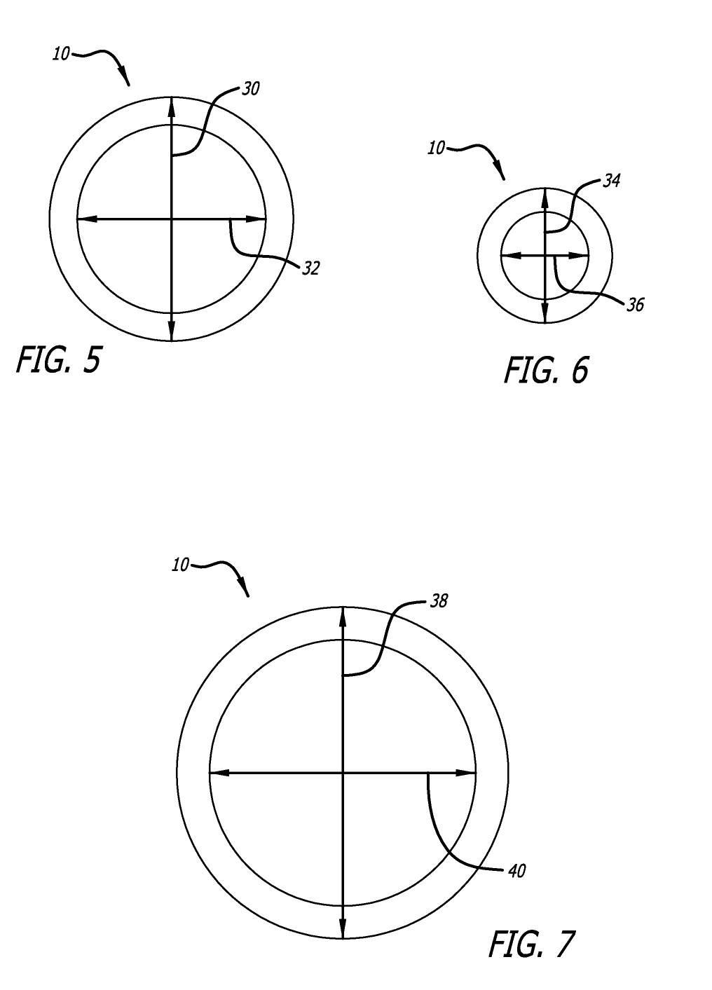

In further keeping with the invention, as shown in FIGS. 5-9, stent 10 has three basic diameters that relate to the Golden Ratio design. As shown in FIG. 5, stent 10 has an outer diameter 30 in the manufactured state, that is, the stent typically is laser cut from a tubular material. The outer diameter 30 can be any number of diameters that are received from a manufacturer from stock tubing to be laser cut into a stent pattern. Typically, the manufactured outer diameter 30 can range from 1.3208 mm (0.052 inch) to 3.175 mm (0.125 inch). These tubing sizes can vary, however, and can be larger or smaller depending on the particular application. The typical diameters as disclosed are suitable for coronary and peripheral arteries and other body lumens that are typically stented. Similarly, a manufactured inner diameter 32 is in the range of about 1.2192 mm (0.048 inch) to 3.0734 mm (0.121 inch). After a manufactured tubing is laser cut to form a stent pattern, the stent is usually electropolished in a chemical solution by well know methods. The electropolishing may reduce the strut radial thickness and strut width dimensions slightly.

As shown in FIG. 6, after the stent 10 is electropolished, it is tightly crimped or compressed onto the balloon portion of a catheter (not shown). The outer diameter 34 and the inner diameter 36 of the stent 10 in the crimped configuration also can range in size depending upon the starting manufactured outer and inner diameters. The crimped outer diameter 34 and the crimped inner diameter 36 of the stent 10 are governed by several factors including the diameter of the deflated balloon upon which the stent is mounted, the size of the lesion and the vessel or artery through which the stent must pass, and the implanted diameter of the stent once the balloon is expanded in the artery.

Referring to FIG. 7, once the stent is implanted in the artery and expanded by the balloon portion of the catheter, the stent 10 has an expanded outer diameter 38 and an expanded inner diameter 40. In the expanded configuration, stent 10 has an expanded outer diameter 38 in the range of 2.10 mm (0.0827 inch) to 5.10 mm (0.2008 inch) and a nominal expanded inner diameter 40 in the range from 2.010 mm (0.0791 inch) to about 5.010 mm (0.1972 inch). Thus, as an example of the application of the Golden Ratio design, a tubular member having a manufactured outer diameter 30 of 1.905 mm (0.05 inch) is implanted in a coronary artery and expanded to a nominal expansion inner diameter 40 of about 3.0 mm (0.1181 inch). Multiplying the Golden Ratio number of 1.618 times the manufactured outer diameter of 1.905 mm (0.075 inch) equals 3.0823 mm (0.1214 inch) which closely approximates the 3.0 mm (0.1181 inch) diameter of the artery in which the stent is implanted.

A list of the dimensions for the various inner and outer diameters, and the Golden Ratio diameter are listed in Table 1. All of the dimensions are in inches except where indicated in mm.

TABLE 1
Initial Tube OD Versus Golden-Ratio Determined Deployed ID

| Initial | Initial | | | | GR |
|---|---|---|---|---|---|
| Tube | Tube | Deployed | Deployed | Deployed | Deployed |
| OD | ID | OD | ID | OD (mm) | ID (mm) |
| 0.052 | 0.048 | 0.0827 | 0.0791 | 2.100 | 2.010 |
| 0.058 | 0.054 | 0.0924 | 0.0888 | 2.347 | 2.256 |
| 0.064 | 0.06 | 0.1021 | 0.0985 | 2.593 | 2.503 |
| 0.07 | 0.066 | 0.1118 | 0.1082 | 2.840 | 2.749 |
| 0.075 | 0.071 | 0.1199 | 0.1163 | 3.045 | 2.955 |
| 0.082 | 0.078 | 0.1312 | 0.1277 | 3.333 | 3.243 |
| 0.088 | 0.084 | 0.1409 | 0.1374 | 3.580 | 3.489 |
| 0.1 | 0.096 | 0.1603 | 0.1568 | 4.073 | 3.982 |
| 0.1125 | 0.1085 | 0.1806 | 0.1770 | 4.587 | 4.496 |
| 0.125 | 0.121 | 0.2008 | 0.1972 | 5.100 | 5.010 |

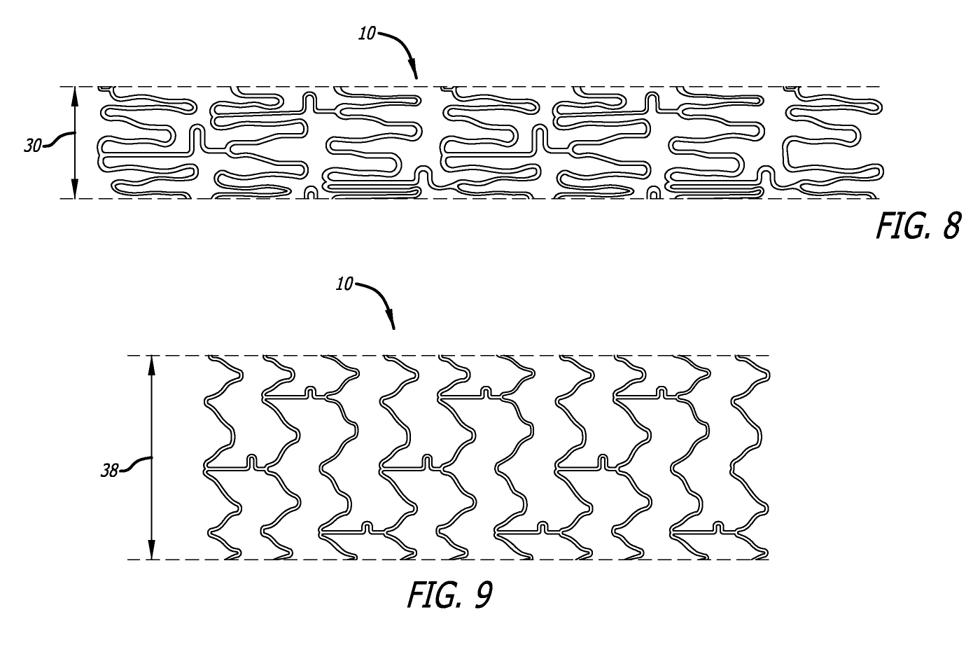

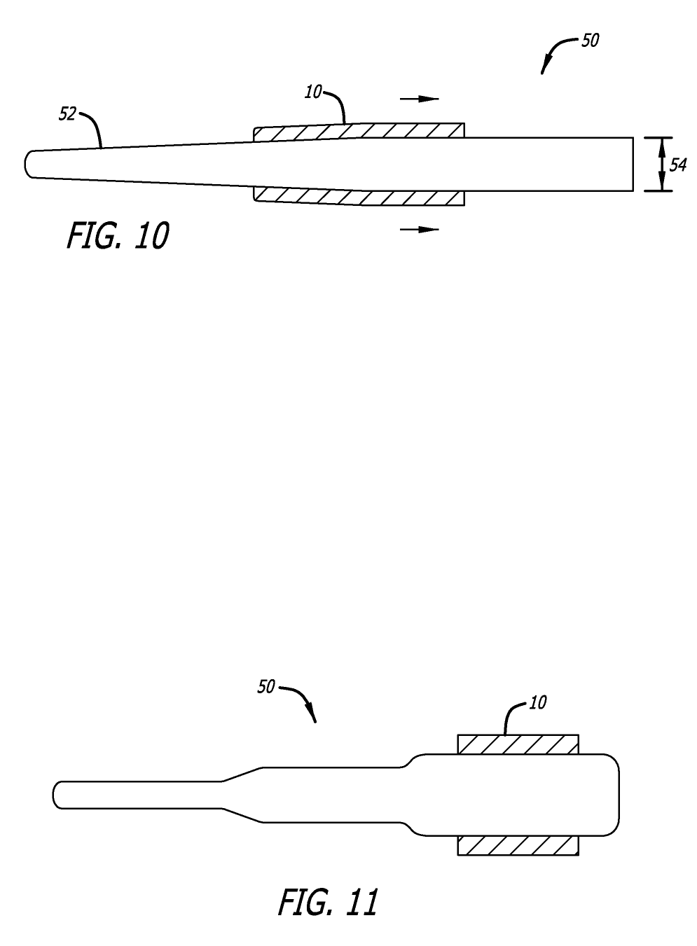

The stent of the present invention can undergo substantial stress when it is crimped from the manufactured diameter onto the balloon portion of a catheter, and then radially expanded by the balloon to the implanted diameter. Most stents, like those shown in FIGS. 8 and 9, undergo substantial stresses in the curved portions of the stent while expanding radially outwardly or being compressed radially inwardly. In order to relieve some of the stresses on the stents during crimping and expansion, and to more easily achieve the Golden Ratio diameter while relieving stress, the stents can be “pre-expanded” and then annealed in order to relieve strain in the curved portions of the stent. In one embodiment, as shown in FIG. 10, a tapered mandrel 50 has a taper 52 at one end and a constant diameter section 54 at the other end. As stent 10 slides over the tapered section 52 it will gradually increase in diameter until it has a constant diameter on the constant diameter section 54. The constant diameter stent is “pre-expanded” and annealed in the pre-expanded state in order to remove stress history within the metallic structure. This provides for a pre-expanded stent that essentially functions as a stent cut from a larger diameter tube. As shown in FIG. 11, an alternative embodiment stepped diameter mandrel 56 operates essentially the same as tapered mandrel 50. The stent 10 slides over the smaller diameters of stepped diameter mandrel 56 until it reaches the greatest diameter and then it is annealed to relieve the stress history within the metallic structure.

The pre-expanded stents shown in FIGS. 10 and 11 can be expanded to diameters greater than those of conventional stents through the reduced levels of expansion strain upon stent deployment. This is achieved since the stents are strain-free at a larger diameter, thereby accumulating less plastic strain as they are expanded. This property is design dependent, however, as some designs (stent patterns) may actually accumulate increased strains during the crimping process. This occurs as pre-expanded stents experience a larger diametric reduction during the crimping process to achieve the same profile as a standard (non-pre-expanded stent), however, expansion strains are generally much higher than those experienced during the crimping process. Thus, there is a net benefit in providing pre-expanded stents. Further, the pre-expanded stents can be designed to have outer and inner diameters that can be customized to more readily achieve the Golden Ratio diameter.

While the invention has been illustrated and described herein in terms of its use as an intravascular stent, it will be apparent to those skilled in the art that the stent can be used in other instances such as to expand body lumens and other vessels in addition to coronary arteries. Other modifications and improvements can be made without departing from the scope of the invention.

What is claimed:

1. An intravascular stent, comprising:
an elongated tubular member having a manufactured inner diameter and a manufactured outer diameter; and
the elongated tubular member further having a nominal expansion inner diameter and nominal expansion outer diameter wherein the nominal expansion outer diameter is approximately 1.618 times larger than the manufactured outer diameter.

2. The intravascular stent of claim 1, wherein the manufactured outer diameter is in the range of 1.3208 mm (0.052 inch) to 3.175 mm (0.125 inch) and the nominal expansion outer diameter is in the range of 2.100 mm (0.0827 inch) to 5.100 mm (0.2008 inch).

3. The intravascular stent of claim 1, wherein the elongated tubular member has a radial thickness in the range of 0.0559 mm (0.0022 inch) to 0.1422 mm (0.0056 inch).

4. The intravascular stent of claim 1, wherein a strut pattern is formed in the elongated tubular member whereby the struts have a width in the range of 0.0457 mm (0.0018 inch) to 0.1067 mm (0.0042 inch).

5. The intravascular stent of claim 1, wherein the elongated tubular member is formed from a metal alloy.

6. The intravascular stent of claim 1, wherein the nominal expansion inner diameter is in the range of 2.0091 mm (0.0791 inch) to 5.0089 mm (0.1972 inch).

7. The intravascular stent of claim 1, wherein the manufactured inner diameter is in the range from 1.2192 mm (0.048 inch) to 3.0734 (0.121 inch).

8. The intravascular stent of claim 1, wherein the elongated tubular member having the manufactured inner and outer diameters are pre-expanded on a mandrel and annealed to provide a stent that can be expanded to a diameter greater than before being pre-expanded and annealed.
